(19) United States
(12) Patent Application Publication
(10) Pub. No.: US 2014/0271505 A1
(43) Pub. Date: Sep. 18, 2014

(54) Methods for Improving Skin Appearance

(71) Applicant: INDERMA, INC., Mill Valley, CA (US)

(72) Inventor: Eva Claiborne, San Rafael, CA (US)

(73) Assignee: Inderma, Inc., Mill Valley, CA (US)

(21) Appl. No.: 13/840,758

(22) Filed: Mar. 15, 2013

(51) Int. Cl. A61K 8/97; A61Q 17/04; A61Q 19/08; A61Q 19/02; A61K 8/64; A61K 8/31

(52) U.S. Cl. CPC A61K 8/97; A61K 8/64; A61K 8/315; A61Q 19/08; A61Q 19/02; A61Q 17/04
USPC 424/59; 424/93.7; 424/62

(57) ABSTRACT

This disclosure concerns method for improving the appearance of skin. The methods includes various steps such as applying a cleanser to an area of skin to cleanse said area of skin; applying an exfoliating treatment to said area of skin; applying a first treatment composition to said area of skin after exfoliating; massaging said first treatment composition on said area of skin until a temperature of said area of skin increases; and applying a second treatment composition to said area of skin.

### BACKGROUND

Changes in skin appearance which result as a person ages can be due to many factors, such as, for example, changes in elastin and collagen. Skin appearance can also be affected by uneven pigment and changes in skin thickness, such as the thinning of the skin under and around the eyes. External factors, such as sun and weather, also play a major role in skin appearance. Moreover, as life expectancy continues to rise and people live longer, there is continued rise in the demand for treatments which improve the appearance of skin and reverse the affects of aging. As the demand for such treatments continue to increase, the skin care and cosmetic market also continues to grow. However, there remains the need for improvement in treatments which improve skin appearance.

### BRIEF SUMMARY

This summary is provided to introduce simplified concepts of methods for improving the appearance or treating skin, such as skin covering the face, neck, shoulders, or chest. Additional details of example methods are further described below in the Detailed Description. This summary is not intended to identify essential features of the claimed subject matter, nor is it intended for use alone in determining the scope of the claimed subject matter.

According to an embodiment, the method may include applying a cleanser to an area of skin to cleanse the area of skin; applying an exfoliating treatment to the area of skin; applying a first treatment composition to the area of skin after exfoliating; massaging the first treatment composition on the area of skin until a temperature of the area of skin increases; and applying a second treatment composition to the area of skin.

According to other embodiments the first treatment composition and the second treatment composition each independently comprises an anti-aging composition, an anti-wrinkle composition, a skin brightening composition, and skin whitening composition or a combination thereof.

### BRIEF DESCRIPTION OF THE DRAWING

The detailed description is set forth with reference to the accompanying figure.

FIG. 1 is a flow diagram of an embodiment of an example process to improve the appearance of, or treatment of an area of skin.

FIG. 2 shows photographs of a subject before and after a first treatment via a method according to the present invention. The pictures show a post treatment reduction in fine lines and a lessening of what were more pronounced wrinkles. The pictures also show a lightening of discolorations on the skin and evening of skin color.

FIG. 3 shows other photographs of a subject before and after treatment with the method according to the present invention. These pictures also show a post treatment reduction in fine lines and a lessening of what were more pronounced wrinkles. The pictures also show a lightening of discolorations on the skin and an evening of skin color.

### DETAILED DESCRIPTION

This disclosure describes embodiments of methods to improve the appearance of, or treatment of skin (such as by reducing the number and appearance of wrinkles and fine lines and/or whitening and/or brightening the skin). The methods according to the present invention may include applying a cleanser to an area of skin to cleanse the area of skin; applying an exfoliating treatment to the area of skin; applying a first treatment composition to the area of skin after exfoliating; massaging the first treatment composition on the area of skin until a temperature of the area of skin increases; and applying a second treatment composition to the area of skin.

According to certain embodiment, any area of skin may be treated with the methods described herein. For example, the area of skin may include skin of the face, neck, shoulder, chest, or a combination thereof. Moreover, the cleanser, exfoliating treatment, first and second treatment composition and other products or compositions used in the methods described herein can be applied to the skin, for example, via hand, cloth, swab, brush, spray or other appropriate techniques.

According to an embodiment, the cleanser can be any cleanser that removes, for example, make-up, excess oil and other unwanted impurities from the skin while also being gentle enough to not damage or overly dry the skin. Exemplary cleansers include but are not limited to clarifying milk cleansers and foaming cleansers. Clarifying milk cleanser include, but are not limited to, Inderma Exfoliating Clarifying Cleanser and other similar cleansers. Moreover, foaming cleansers include, but are not limited to, Inderma Foamy Cleanser and other similar cleansers.

According to an embodiment, the exfoliating treatment may include applying a peptide crystal composition to the area of skin, applying a chemical exfoliating composition to the area of skin, or both. According to certain embodiments, the peptide crystal composition comprises at least one peptide complex that reduces release of acetylcholine and an abrasive material. Moreover, according to other embodiments, the chemical exfoliating composition comprises at least one monohydroxybenzoic acid, at least one alpha hydroxyl acid, or both. Useful exfoliating peptide crystal compositions include, but are not limited to, polyethylene. Likewise, useful chemical exfoliating compositions include, but are not limited to glycolic acid, lactic acid, malic acid, and tartaric acid based compositions. According to certain embodiments, substantially all of the cleanser is removed prior to exfoliating.

According to certain embodiments, the massage step may be from 5 minutes to about 30 minuted in length, from about 10 minutes to about 25 minutes in length or from about 15 minutes to about 20 minutes in length. Moreover, during the massage step, the temperature of the area of skin may increase from about 0.5° F. to about 5° F., from about 1° F. to about 3° F., or from about 1° F. to about 2° F. According to other embodiments, skin temperature may be increased more than 0.25° F., more than 0.5° F., more than 1° F., more than 2° F., more than 3° F., more than 4° F. or even greater. The increase in temperature may be measured by, for example, a thermomter or other temperature detecting device. According to certain embodiments, massaging my be accomplished with the use of a person's hands, fingers, elbows, or other part of the body of the person performing the massage. In other embodiments, massaging may be accomplished with the use of a massage device. Acceptable massage device may include Clarisonic hand held and/or hand held light devices.

According to certain embodiment, the method may include applying a mask and/or a moisturizer to the area of skin after massaging to hydrate the area of skin. Mask products that may be used in the present methods include, but are not limited to creamy or gel based masks. Moreover, moisturizers that may be used in the method include, but are not limited to, formulations having hydrating, regenerating, nourishing, rejuvenating, and/or illuminating properties.

According to an embodiment, the first treatment composition and the second treatment composition may each independently comprise an anti-aging composition, an anti-wrinkle composition, a skin brightening composition, a skin whitening composition or a combination thereof. According to certain embodiments, the anti-aging composition may include at least one collagen stimulating peptide; at least one anti-inflammatory compound; and at least one skin tightening agent. According to certain embodiments, the anti-wrinkle composition may include at least one fluorinated perfluorocarbon; stem cells from at least one plant species; and at least one peptide complex that reduces release of acetylcholine. According to still other embodiments, the skin-brightening or whitening composition may include two or more tyrosinase inhibiting compounds; one or more extracts from at least one plant species to inhibit alpha-Melanoctye-stimulating hormone; and one or more cosmetically acceptable carriers. In yet other embodiments, the skin lightening or brightening composition may include at least one peptide having tyrosinase inhibiting qualities; at least on hydroquinone derivative; at least one extract from at least one plant species; and a cosmetically acceptable carrier.

According to other embodiments, the method may also comprise applying a sun protection composition to the area of skin after said second treatment composition is applied. Useful sun protection compositions include, but are not limited to, products containing, for example, organic chemicals that absorb or reflect ultraviolet light, zinc oxide, titanium dioxide, octinoxate, and/or chromophores that scatter/absorb/reflect ultraviolet light.

FIG. 1 is a flow diagram of an embodiment of an example method 100 to improve the appearance of or treat skin. At 102, the method 100 includes applying a cleanser to an area of skin. According to certain embodiment, the cleanser may be a foaming cleanser or milk cleanser. Moreover, the area of skin may include facial skin, neck skin, shoulder skin, chest skin or a combination thereof.

At 104, the method 100 includes applying an exfoliating treatment to the area of skin. The exfoliating treatment may include applying a peptide crystal composition as described above to the area of skin, applying a chemical exfoliating composition as described above to said area of skin, or both. For example, an exemplary exfoliation treatment may include applying a peptide crystal composition and then applying a chemical exfoliating treatment.

At 106, the method 100 includes applying a first treatment composition to the area of skin. The first treatment composition may include applying to the area of skin an anti-aging composition, an anti-wrinkle composition, a skin brightening composition, a skin whitening composition or a combination thereof as described above.

At 108, the method 100 includes massaging the first treatment composition the area of skin until the temperature of the area of skin increases. As described above, the massage step may be from 5 minutes to about 30 minuted in length, from about 10 to about 25 minutes in length or from about 15 to about 20 minutes in length. According to certain embodiments, massaging my be accomplished with the use of a person's hands, fingers, elbows, or other part of the body. In other embodiments, massaging may be accomplished with the use of a massage device.

At 110, the method 100 includes applying a second composition to the area of skin. The second composition may include applying to the area of skin an anti-aging composition, an anti-wrinkle composition, a skin brightening composition, a skin whitening composition or a combination thereof as described above.

While not shown in FIG. 1, after the massaging and prior to applying the second treatment, the method may include applying a moisturizer and/or applying a toner.

It should be understood that the method steps particularly mentioned above are merely examples and that some embodiments of methods may include additional steps as desired. Generally in the practice of methods of the invention, the compositions or treatment are topically applied to the skin on a subject in need of treatment in a predetermined or as-needed regimen either at intervals, it generally being the case that gradual anti-wrinkle and/or anti-aging is noted with each successive application of the methods described herein. According to a certain embodiment, the first and second treatments include applying a treatment composition (first or second) in one form (e.g. a serum) followed by applying the same or a different treatment composition according to the present invention in another form (e.g. a cream).

### EXAMPLES

Certain embodiments of the present invention now will be illustrated by the following Examples. The present invention may, however, be embodied in many different forms and should not be construed as limited to the embodiments set forth herein; rather, these embodiments are provided so that this disclosure will be thorough and complete, and will fully convey the scope of the invention to those skilled in the art.

### Example

In this example, a subject was treated with a method according the present disclosure. Before and after photographs are included as FIGS. 1 and 2.

First, the subject's face was cleansed with a cleanser.

The cleanser included the following ingredients: Aloe Barbadensis Leaf, Purified Water, Hydrogenated Polyisobutene, Glycerin, Cetyl Ethylhexanoate, Polysorbate-20, Glyceryl Stearate, PEG-100 Stearate, Sorbitol, Caprylic/Capric Triglyceride, Simmondsia Chinensis (Jojoba) Seed Oil, Decyl Glucoside, Mandelic Acid, Stearic Acid, Vitis Vinifera (Grape) Seed Oil, Iodopropynyl Butylcarbamate, Cetyl Alcohol, Diazolidinyl Urea, Carbomer, Triethanolamine, Butylene Glycol, Camellia Oleifera (Green Tea) Leaf Extract, Chamomilla Recutita (Matricaria) Flower Extract, and Fragrance.

After the cleanser was removed with a wet towel, a peptide crystal exfoliating composition was applied to the subject's face with care taken to avoid the eye area. A chemical exfoliating composition was then applied to the subject's face on top of the peptide crystal exfoliation composition. Both the peptide crystal exfoliating composition and the chemical exfoliating composition were left on the subject's face for about 5-10 minutes wherein a white foam appeared on the subject's face. Both compositions were then removed a wet towel.

The peptide crystal exfoliation composition included the following ingredients: Caprylic/Capric Triglyceride, Aluminum Oxide, Sodium Bicarbonate, Glyceryl Stearate SE, Oleth-10, Glycerin, Polysorbate-60, Caprylyl Glycol, Phenoxyethanol, Acetyl Hexapeptide-8, Fragrance, Sorbitol, Magnesium Ascorbyl Phosphate, Retinyl Palmitate, Tocopheryl Acetate.

The chemical exfoliating composition included the following ingredients: Purified Water, Lactic Acid, Hydroxyethylcellulose, Butylene Glycol, Methyldibromo Glutaronitrile, Methylchloroisothiazolinone, Methylisothiazolinone, Phenoxyethanol, Salicylic Acid.

The subject's face was then cleansed to remove any residual peptide crystal exfoliation composition and chemical exfoliating composition with the cleanser described above.

A toner was then sprayed on the subject's face to neutralize and remove any traces of the cleanser.

The toner included the following ingredients: Aloe Barbadensis Leaf, Purified Water, Glycerin, Hyaluronic Acid, Malus Domestica Fruit Cell Culture Extract, Xanthan Gum, Glycerin, Lecithin, Phenoxyethanol, Caprylyl Glycol, Polysorbate-20, Butylene Glycol, Chamomilla Recutita (Matricaria) Flower Extract, Helianthus Annuus (Sunflower) Seed Oil, Calendula Officinalis Flower Extract and Fragrance.

A skin lightening serum followed by a skin lightening cream were then applied to the subject's face. The subject's face was then massaged for about 20-minutes. The skin temperature of the subject face increased about 1.1-1.5° F.

The skin lightening serum included the following ingredients: Aloe Barbadensis Leaf, Purified Water, Glycerin, Phospholipid, Arbutin, Linoleic Acid, Linolenic Acid, Tocopheryl Acetate, Ascorbyl Palmitate, Glutathione, Alcohol, Olea Europaea (Olive) Fruit Oil, Ethylhexyl Palmitate, Polyacrylamide, C13-14 Isoparaffin, Laureth-7, Hydrolyzed Beta Maritima Extract, Malus Domestica Fruit Cell Culture Extract, Xanthan Gum, Lecithin, Phenoxyethanol, Caprylyl Glycol, Glycyrrhiza Uralensis (Licorice) Root Extract, Caprylic/Capric Triglyceride, Hyaluronic Acid, Bellis Perennis (Daisy) Flower Extract, Salicyloyl Phytosphingosine, Simmondsia Chinensis (Jojoba) Seed Oil, Ceramide 3, Sphingolipids, Dimethyl Methoxyl Chromanyl Palmitate, and Fragrance(s).

The skin lightening cream included the following ingredients: Aloe Barbadensis Leaf, Purified Water, Glycerin, Lepidium Sativum Sprout Extract, Lecithin, Glycerin, Phenoxyethanol, Polyacrylamide, C13-14 Isoparaffin, Laureth-7, Olea Europaea (Olive) Fruit Oil, Ethylhexyl Palmitate, Arbutin, Caprylic/Capric Triglyceride, Malus Domestica Fruit Cell Culture Extract, Xanthan Gum, Lecithin, Hyaluronic Acid, Hydrolyzed Beta Maritima Extract, Butylene Glycol, Glycyrrhiza Uralensis (Licorice) Root Extract, Caprylyl Glycol, Tocopheryl Acetate, Bellis Perennis (Daisy) Flower Extract, Salicyloyl Phytosphingosine, Ceramide 3, and Fragrance.

A mask was then applied to the subjects face and left in place for about 15 to 20 minutes. The mask was then removed with wet towels.

The mask included the following ingredients: Aloe Barbadensis Leaf, Purified Water, Stearic Acid, Glyceryl Stearate, PEG-100 Stearate, Squalene, Cetyl Alcohol, Pentylene Glycol, Titanium Dioxide, Caprylic/Capric Triglyceride, Hydrogenated Polydecene, Sodium Palmitoyl Proline, Nymphaea Alba Flower Extract, Caprylyl Glycol, Phyenoxyethanol, Prunus Armeniaca (Apricot) Kernel Oil, Helianthus Annuus (Sunflower) Seed Oil, Potassium Sorbate, Yeast Extract, Triethanolamine, Vitis Vinifera (Grape) Seed Oil, Chamomilla Recutita (Matricaria) Flower Extract, Fragrance, Disodium EDTA.

The toner described above was then again sprayed on the subject's face to neutralize and remove any traces of the mask.

The lightening serum described above was then again applied to the subject's face.

As shown in FIGS. 2 and 3, the dramatic softening of the melasma blotch(es) after only one treatment with the method described above. Additionally, the subject's skin had a healthy glow that looked hydrated and smoother. Applying previously known skin care regimens alone, it would take weeks to see the results that occurred with the method described herein.

While applicant's disclosure has been provided with reference to specific embodiments above, it will be understood that modifications and alterations in the embodiments disclosed may be made by those practiced in the art without departing from the spirit and scope of the invention. All such modifications and alterations are intended to be covered.

We claim:

1. A method for treating skin comprising:
a. applying a cleanser to an area of skin to cleanse said area of skin;
b. applying an exfoliating treatment to said area of skin;
c. applying a first treatment composition to said area of skin after exfoliating;
d. massaging said first treatment composition on said area of skin until a thermometer shows a temperature increase of said area of at least 0.25° F.; and
e. applying a second treatment composition to said area of skin.

2. The method according to claim 1, wherein said first treatment composition and said second treatment composition each independently comprises an anti-aging composition, an anti-wrinkle composition, a skin brightening composition, a skin whitening composition or a combination thereof.

3. The method according to claim 1, wherein said exfoliating treatment comprises:
applying a peptide crystal composition to said area of skin, applying a chemical exfoliating composition to said area of skin, or both.

4. The method according to claim 3, wherein said peptide crystal composition comprises at least one peptide complex that reduces release of acetylcholine and an abrasive material.

5. The method according to claim 3, wherein said chemical exfoliating composition comprises at least one monohydroxybenzoic acid, at least one alpha hydroxyl acid, or both.

6. The method according to claim 1, wherein during said massaging the temperature of said area of skin is increased from about 0.5° F. to about 5° F.

7. The method according to claim 6, wherein during said massaging the temperature of said area of skin is increased from about 1° F. to about 2° F.

8. The method according to claim 1, wherein substantially all of said cleanser is removed prior to exfoliating.

9. The method according to claim 1, further comprising applying a mask to said area of skin after said massaging to hydrate said area of skin.

10. The method according to claim 1, further comprising applying a moisturizer to said area of skin after said massaging.

11. The method according to claim 1, wherein said first treatment composition is an anti-aging composition comprising:
a. at least one collagen stimulating peptide;
b. at least one anti-inflammatory compound; and
c. at least one skin tightening agent.

12. The method according to claim 1, wherein said first treatment composition is an anti-wrinkle composition comprising:
a. at least one fluorinated perfluorocarbon;
b. stem cells from at least one plant species; and
c. at least one peptide complex that reduces release of acetylcholine.

13. The method according to claim 1, wherein said first treatment composition is a skin brightening or lightening composition comprising:
a. two or more tyrosinase inhibiting compounds;
b. one or more extracts from at least one plant species to inhibit alpha-melanocyte stimulating hormone, and
c. one or more cosmetically acceptable carriers.

14. The method according to claim 1, wherein said first treatment composition is a skin lightening composition comprising:
a. at least one peptide having tyrosinase inhibiting activity;
b. at least one hydroquinone derivative;
c. at least one extract from at least one plant species; and
d. a cosmetically acceptable carrier.

15. The method according to claim 1, wherein said cleanser is a foaming cleanser or a milk cleanser.

16. The method according to claim 1, further comprising applying a sun protection composition to said area of skin after said second treatment composition is applied.

17. The method according to claim 1, wherein said area of skin comprises skin located on the face, neck, back, shoulder, chest, or a combination thereof.
